(19) United States
(12) Patent Application Publication
(10) Pub. No.: US 2026/0201082 A1
(43) Pub. Date: Jul. 16, 2026

(54) POLYMER MATERIAL

(71) Applicant: NATIONAL INSTITUTE OF ADVANCED INDUSTRIAL SCIENCE AND TECHNOLOGY, Chiyoda-ku, Tokyo (JP)

(72) Inventors: Kimio SUMARU, Tsukuba-shi, Ibaraki (JP); Toshiyuki TAKAGI, Tsukuba-shi, Ibaraki (JP)

(21) Appl. No.: 19/135,781

(22) PCT Filed: Dec. 1, 2023
(86) PCT No.: PCT/JP2023/043184

§ 371 Date: Jun. 4, 2025

(30) Foreign Application Priority Data
Dec. 9, 2022 (JP) 2022-197076

(51) Int. Cl. C08F 8/30; C08G 81/02; C08J 3/28; C12N 5/071; C12N 5/09; G03F 7/038

(52) U.S. Cl. CPC C08F 8/30; C08G 81/025; C08J 3/28; C12N 5/0686; C12N 5/0693; G03F 7/038; C08F 2810/20; C08J 2329/04; C08J 2387/00; C12N 2533/30; C12N 2537/10

(57) ABSTRACT

Provided is a polymer material including a hydroxyl group-containing polymer which is acetalized with a compound represented by Formula (1), in which the hydroxyl group-containing polymer contains two or more hydroxyl groups in a main chain and/or a side chain, and the polymer material forms a crosslinked substance by irradiation with light.

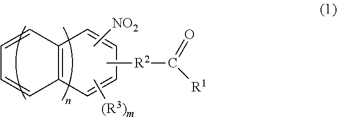

[In the formula, R¹ represents a hydrogen atom or the like, R² represents a single bond or the like, and R³ represents a hydroxyl group or the like. n represents an integer of 0 or 1 representing a monocycle or a fused ring. m represents the number of R³ bonded to an aromatic ring, m represents an integer of 0 to 4 in a case where n represents 0, and m represents an integer of 0 to 6 in a case where n represents 1.]

### TECHNICAL FIELD

The present invention relates to a polymer material.

### BACKGROUND ART

Various polymer materials that exhibit a change in physical properties due to light have been developed so far. A material that is dissolved in a solvent before light irradiation and is insolubilized in the solvent after a photoreaction is widely put into practical use as a negative tone photoresist, but there are few practical use examples of a material that is insolubilized by a photoreaction in an aqueous system. Although there are practical examples of a material cured by a photopolymerization initiator (for example, aqueous UV polymers O-106 and O-391, manufactured by CHUKYO YUSHI CO., LTD., and FOM-03011, manufactured by FUJIFILM Wako Pure Chemical Corporation), these materials contain a photopolymerization initiator or the like of low molecular weight molecules (a photoradical generator, a photoacid generator, or a photobase generator) and thus have a problem in that the molecules derived from the photopolymerization initiator are liberated from an insoluble substance generated by photocrosslinking or the like.

As a water-soluble polymer that does not contain such a low molecular weight compound, polyvinyl alcohol incorporating stilbazolium has been developed (Non-Patent Literature 1), and biological applications and the like have been examined (Non-Patent Literature 2). However, since insolubilization by photocrosslinking requires light energy of several tens of J/cm², there is a problem that exposure takes a considerable amount of time. In addition, it has been reported that the polyvinyl alcohol incorporated with uracil is also photocrosslinked, but the insolubilization requires light having a short wavelength of less than 300 nm (Non-Patent Literature 3).

As another water-soluble polymer containing no low molecular weight compound, a product of a photocrosslinking type using an azide-based molecule has been commercialized (for example, BIOSURFINE (registered trademark)-AWP, manufactured by Toyo Gosei Co., Ltd.), and there is also a report that photoresponsive crosslinking has been realized by incorporating diazirine as a photoresponsive molecule similar to azide into a protein through an active ester group (Non-Patent Literature 4). However, it is considered that the crosslinking requires a considerable amount of light energy of greater than 10 J/cm².

As another photocrosslinking mechanism, a technique of inactivating (“caging”) an active site with bonding properties by bonding a nitrobenzyl compound in advance has also been examined (Non-Patent Literature 5). The caging is released by irradiation with light at an appropriate timing to restore the activity, and crosslinking is formed by bonding to a counterpart residue, but it is necessary to create a considerably complicated structure to realize this, and there is no report on the photocrosslinking of a polymer based on this principle.

As described above, many polymer materials that are photocrosslinked in an aqueous system have a problem in that a liberatable low molecular weight component is contained as an additive, and there is a practical problem that a large amount of light energy is required for crosslinking and ultraviolet rays having a short wavelength of less than 300 nm is required.

Patent Literature 1 discloses a polymer having a nitrobenzaldehyde derivative in a side chain, and the same polymer changes from an insoluble state in water to a soluble state in water by irradiation with light. However, according to Patent Literature 1, in a case where the same polymer is irradiated with light in a solid phase state, for example, in the air or in a poor solvent, crosslinking is caused by equilibrium dimerization or the like of a nitroso group, and solubility in a solvent is decreased.

### CITATION LIST

### Patent Literature

[Patent Literature 1] International Publication No. WO2017/213226

### Non-Patent Literature

[Non-Patent Literature 1] Journal of Photopolymer Science and Technology, Vol. 8, No. 1, 1995, pp. 125 to 128
[Non-Patent Literature 2] Journal of Polymer Science Part A: Polymer Chemistry Vol. 20, 1982, pp. 1411 to 1417
[Non-Patent Literature 3] Journal of Photopolymer Science and Technology, Vol. 14, No. 2 2001, pp. 295 and 296
[Non-Patent Literature 4] J Mater Chem. 2012 Oct. 7; 22 (37): pp. 19429 to 19437
[Non-Patent Literature 5] Jurgen Stampfl, Robert Liska, and Aleksandr Ovsianikov, “Multiphoton Lithography: Techniques, Materials and Applications, First Edition” Wiley-VCH Verlag GmbH & Co. KGaA, 2017, p. 197

### SUMMARY OF INVENTION

### Technical Problem

An object of the present invention is to provide a polymer material which can be crosslinked even with ultraviolet rays (UVA) having a long wavelength of 320 nm or greater, does not require the addition of a low molecular weight compound involved in a photoreaction, which can be liberated after crosslinking, and can be crosslinked in a liquid phase state.

### Solution to Problem

The present invention provides the following aspects [1] to [18].

[1] A polymer material including: a hydroxyl group-containing polymer which is acetalized with a compound represented by Formula (1), in which the hydroxyl group-containing polymer contains two or more hydroxyl groups in a main chain and/or a side chain, the acetalization is based on a reaction between a group represented by —R²—CR¹O in General Formula (1) and two hydroxyl groups in the hydroxyl group-containing polymer, and the polymer material forms a crosslinked substance by irradiation with light.

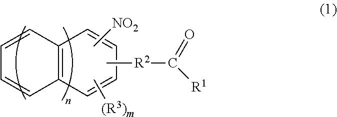

[In the formula, R¹ represents a hydrogen atom, a methyl group, or a cyclopropyl group, R² represents a single bond, an alkylene group having 1 to 3 carbon atoms, an alkenylene group having 2 or 3 carbon atoms, or a group represented by —C(CR¹O)—, and R³ each independently represents an alkyl group having 1 to 3 carbon atoms, a hydroxyalkyl group having 1 to 3 carbon atoms, a hydroxyl group, a halogen atom, an alkoxycarbonyl group, a nitro group, or an arylsulfonyl group. n represents an integer of 0 or 1 representing a monocycle or a fused ring. m represents the number of R³ bonded to an aromatic ring, and m represents an integer of 0 to 4 in a case where n represents 0, and m represents an integer of 0 to 6 in a case where n represents 1. Here, the nitro group in the formula is adjacent to the group represented by —R²—CR¹O or is adjacent to R³ which represents an alkyl group having 1 to 3 carbon atoms.] Here, in a case where a plurality of R¹ is present, R¹ may be the same as or different from each other.

[2] The polymer material according to [1], wherein the compound represented by Formula (1) is at least one of the compounds represented by the following formulae (in the formulae, X represents a halogen atom or a methyl group).

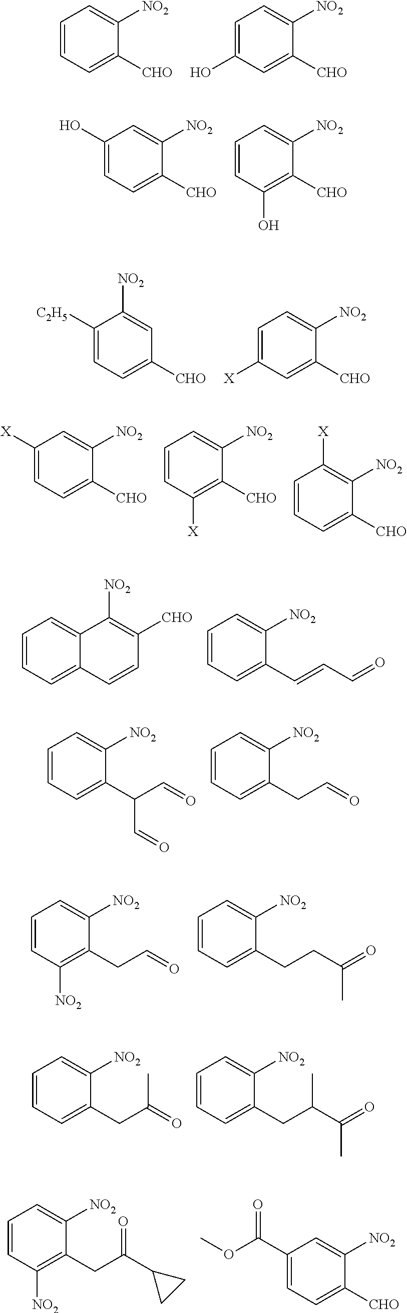

[3] The polymer material according to [1] or [2], wherein R¹ represents a hydrogen atom, R² represents a single bond, an alkylene group having 1 to 3 carbon atoms, or an alkenylene group having 2 or 3 carbon atoms, R³ represents a hydroxyl group, a methyl group, or an ethyl group, n represents 0, and m represents an integer of 0 to 4.

[4] The polymer material according to any one of [1] to [3], wherein the compound represented by Formula (1) is at least one of the compounds represented by the following formulae.

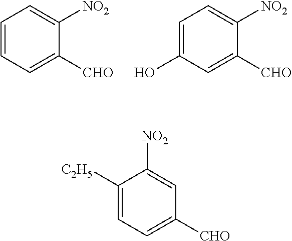 [SMILES: O=Cc1ccccc1[N+](=O)[O-] ; O=Cc1cc(O)ccc1[N+](=O)[O-] ; CCc1ccc(C=O)cc1[N+](=O)[O-]]

[5] The polymer material according to any one of [1] to [4], wherein the hydroxyl group-containing polymer is at least one of the following polymers (a), (b), and (c), or a polymer having at least one of a carboxy group and a sulfonic acid group (the carboxy group and the sulfonic acid group may form a salt) in the polymer.
(a) Polyvinyl alcohol
(b) At least one vinyl alcohol copolymer selected from a linear vinyl alcohol copolymer and a graft vinyl alcohol copolymer
(c) At least one glycerol polymer selected from linear polyglycerol, hyperbranched glycerol, glycerol dendron, and glycerol dendrimer

[6] The polymer material according to [5], wherein the vinyl alcohol copolymer (b) is polyethylene glycol-graft-polyvinyl alcohol.

[7] The polymer material according to [1], wherein the hydroxyl group-containing polymer is polyvinyl alcohol or polyethylene glycol-graft-polyvinyl alcohol, the compound represented by the Formula (1) is at least one of the compounds represented by the following formulae, and, wherein in a state of a solution in a solvent comprising water and/or a water-miscible organic solvent, a water-insoluble gel is generated from the solution state upon irradiation with ultraviolet rays, and in a solid state, the solid becomes insoluble in the solvent upon irradiation with ultraviolet rays.

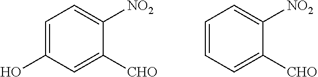 [SMILES: O=Cc1cc(O)ccc1[N+](=O)[O-] ; O=Cc1ccccc1[N+](=O)[O-]]

[8] The polymer material according to [1], wherein the hydroxyl group-containing polymer is polyvinyl alcohol containing a carboxy group or a sulfonic acid group (the carboxy group and the sulfonic acid group may form a salt), and the compound represented by Formula (1) is a compound represented by the following formula, and wherein a gel is generated by irradiating with ultraviolet rays in a solution state of a solvent containing at least one of water, a buffer solution, and a base that forms a salt of the carboxy group or the sulfonic acid group.

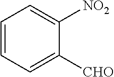 [SMILES: O=Cc1ccccc1[N+](=O)[O-]]

[9] The polymer material according to [1], wherein the hydroxyl group-containing polymer is polyvinyl alcohol containing a carboxy group or a sulfonic acid group (the carboxy group and the sulfonic acid group may form a salt), and the compound represented by Formula (1) is a compound represented by the following formula, and wherein a gel is generated by irradiating with ultraviolet rays in a state where the polymer material is dissolved in an aqueous solution containing a base that forms a salt of the carboxy group or the sulfonic acid group.

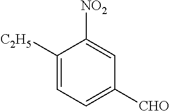 [SMILES: CCc1ccc(C=O)cc1[N+](=O)[O-]]

[10] The polymer material according to [1], wherein the hydroxyl group-containing polymer is polyvinyl alcohol which is acetalized with the compound represented by Formula (1) and a compound containing a formyl group and a carboxy group, and the compound represented by Formula (1) is a compound represented by the following formula, and wherein a gel is generated by irradiating with ultraviolet rays in a state where the polymer material is dissolved in an aqueous solution containing a base that forms a salt of the carboxy group.

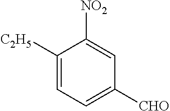 [SMILES: CCc1ccc(C=O)cc1[N+](=O)[O-]]

[11] The polymer material according to [10], wherein the aqueous solution further contains cultured cells and a liquid culture medium for cell culture.

[12] A liquid material wherein the polymer material according to any one of [1] to [11] is dissolved, dispersed, or swollen.

[13] A photolithography material including: the polymer material according to any one of [1] to [11].

[14] A crosslinked substance which is obtained by irradiating the polymer material according to any one of [1] to [11] with ultraviolet rays.

[15] A cell adhesion inhibitor including: a crosslinked substance obtained by irradiating the polymer material according to any one of [7] to [11] with ultraviolet rays.

[16] A cell culture gel including: a crosslinked substance obtained by irradiating the polymer material according to [10] or [11] with ultraviolet rays.

### Advantageous Effects of Invention

According to the present invention, it is possible to provide a polymer material which can be crosslinked even with ultraviolet rays (UVA) having a long wavelength of 320 nm or greater, does not require the addition of a low molecular weight compound involved in a photoreaction, which can be liberated after crosslinking, and can be crosslinked in a liquid phase state.

### BRIEF DESCRIPTION OF DRAWINGS

FIG. 1 shows a state of OHNBA-PVA of Example 1 after being subjected to pattern irradiation with ultraviolet rays, immersed in water, and washed with 70% ethanol.

FIG. 2 is an image showing a state of the OHNBA-PVA of Example 2 after being subjected to pattern irradiation with ultraviolet rays and immersed in water.

FIG. 3 is an image showing a state of OHNBA-PVAPEG of Example 3 after being subjected to pattern irradiation with ultraviolet rays and washed with water.

FIG. 4 is an image showing cell patterning formed by performing pattern irradiation on OHNBA-PVAPEG of Example 3 with ultraviolet rays, washing the OHNBA-PVAPEG with water, and seeding MDCK cells.

FIG. 5 is an image showing a state of NBA-PVAPEG of Example 5 after being subjected to pattern irradiation with ultraviolet rays and washed with water.

FIG. 6 is an image showing a state of NBA-PVAPEG of Example 5 after being subjected to pattern irradiation with ultraviolet rays, placed in a high-humidity environment, and washed with water.

FIG. 7 is an image showing a gel remaining after NBA-PVA of Example 7 is subjected to pattern irradiation with ultraviolet rays and washed with water.

FIG. 8 is an image showing that a gel obtained from NBA-FBA-PVA of Example 11 can be taken in hand.

FIG. 9 is an image showing a gel obtained from ENBA-GoA-PVA of Example 12.

FIG. 10 is an image showing a gel obtained by immersing a gel obtained from ENBA-GoA-PVA of Example 12 in water to swell the gel.

FIG. 11 is an image showing that a gel obtained by immersing a gel obtained from ENBA-GoA-PVA of Example 12 in water to swell the gel and setting the water content thereof to about 99.8% can be taken in hand.

FIG. 12 is an image showing HeLa cells cultured in a photocrosslinked ENBA-GoA-PVA gel obtained from ENBA-GoA-PVA of Example 12.

FIG. 13 is an image showing HeLa cells cultured in a photocrosslinked ENBA-GoA-PVA gel obtained from ENBA-GoA-PVA of Example 12.

FIG. 14 is an image showing HeLa cells cultured in a photocrosslinked ENBA-GoA-PVA gel obtained from ENBA-GoA-PVA of Example 12.

FIG. 15 is an image showing HeLa cells cultured in a photocrosslinked ENBA-GoA-PVA gel obtained from ENBA-GoA-PVA of Example 12.

FIG. 16 is a graph showing a relationship between an applied load and a relative cross-sectional area of a test piece (φ6 mm×1.5 mm) of a gel obtained by photo sol-gel transition of a 1 wt % solution of ENBA-GoA-PVA of Example 13.

FIG. 17 is a stress-strain curve obtained by repeatedly performing a compression test twice on a test piece (φ6 mm×1.5 mm) of a gel obtained by photo sol-gel transition of a 1 wt % solution of ENBA-GoA-PVA of Example 13.

FIG. 18 shows images of an adhesion test of a photocrosslinked gel obtained from ENBA-GoA-PVA of Example 13. FIG. 18(a) is an image showing a state in which an ENBA-GoA-PVA solution is dropped onto an adherend, FIG. 18(b) is an image showing a state in which the adherend is covered with another adherend, FIG. 18(c) is an image showing a state in which the adherend is irradiated with ultraviolet rays, and FIG. 18(d) is an image showing a state in which a weight is suspended.

FIG. 19 is a graph showing swelling properties of a gel in an aqueous solution, which is obtained by photo sol-gel transition of an ENBA-GoA-PVA aqueous solution of Example 13.

### DESCRIPTION OF EMBODIMENTS

A polymer material according to the present invention is a polymer material containing a hydroxyl group-containing polymer which is acetalized with a compound represented by Formula (1) (hereinafter, also abbreviated as “compound 1” in some cases, and the same applies to other compounds), and the polymer material forms a crosslinked substance by irradiation with light. Further, the hydroxyl group-containing polymer contains two or more hydroxyl groups in a main chain and/or a side chain, the acetalization is based on a reaction between a group represented by —R²—CR¹O in General Formula (1) and two hydroxyl groups in the hydroxyl group-containing polymer.

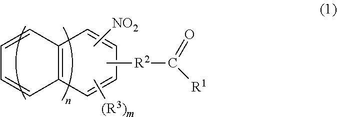

[In the formula, R¹ represents a hydrogen atom, a methyl group, or a cyclopropyl group, R² represents a single bond, an alkylene group having 1 to 3 carbon atoms, an alkenylene group having 2 or 3 carbon atoms, or a group represented by —C(CR¹O)—, and R³ each independently represents an alkyl group having 1 to 3 carbon atoms, a hydroxyalkyl group having 1 to 3 carbon atoms, a hydroxyl group, a halogen atom, an alkoxycarbonyl group, a nitro group, or an arylsulfonyl group. n represents an integer of 0 or 1 representing a monocycle or a fused ring. m represents the number of R³ bonded to an aromatic ring, and m represents an integer of 0 to 4 in a case where n represents 0, and m represents an integer of 0 to 6 in a case where n represents 1. Here, the nitro group in the formula is adjacent to the group represented by —R²—CR¹O or is adjacent to R³ which represents an alkyl group having 1 to 3 carbon atoms.] Here, in a case where a plurality of R¹ is present, R¹ may be the same as or different from each other.

The compound 1 is represented by General Formula 1a in a case where n represents 0, and is represented by General Formula 1b in a case where n represents 1. In a case where n represents 0, it is possible to obtain unsubstituted R³ in which m represents 0 to tetra-substituted R³ in which m represents 4. Meanwhile, in a case where n represents 1, it is possible to obtain unsubstituted R³ in which m represents 0 to hexa-substituted R³ in which m represents 6.

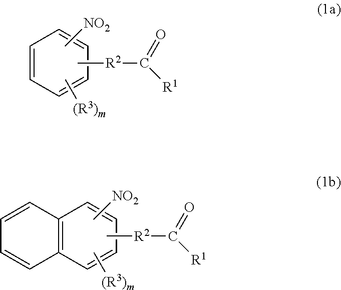

The polymer material is a hydroxyl group-containing polymer acetalized with the compound 1, but in a case where the hydroxyl group-containing polymer is represented by Chemical Formula (2a), the hydroxyl group-containing polymer acetalized with the compound 1 can be represented by Chemical Formula (10a). R¹, R², R³, m, and n each have the same definition as described above (the same applies hereinafter). Further, in the present invention, “acetalization” includes both a reaction for obtaining an acetal derived from an aldehyde and a reaction for obtaining a ketal derived from a ketone.

In addition, the acetalization only needs to be performed by reacting the compound 1 with a hydroxyl group-containing polymer, for example, under acidic conditions. The compound represented by Chemical Formula (2a) is an example of a hydroxyl group-containing polymer, and the compound represented by Chemical Formula (10a) is an example of a hydroxyl group-containing polymer acetalized by Chemical Formula 1.

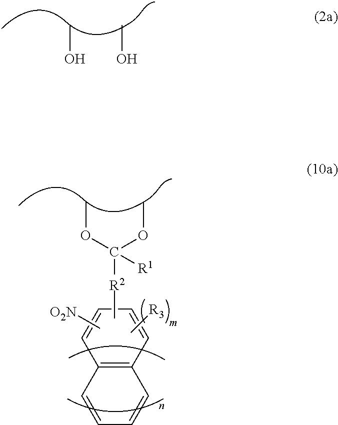

Although two hydroxyl groups are shown in Chemical Formula (2a) and only one acetal bond is shown in Chemical Formula (10a), three or more hydroxyl groups in Chemical Formula (2a) and two or more acetal bonds in Chemical Formula (10a) can be present. All or some of the plurality of hydroxyl groups in Chemical Formula (2a) may be acetalized. The proportion of the hydroxyl groups in the hydroxyl group-containing polymer to be acetalized can be set to 50% to 100% by mole. Further, in Chemical Formula (2a), it is preferable that two or more hydroxyl groups are present at adjacent positions or positions separated by two atoms (that is, hydroxyl groups are bonded to an atom adjacent to an atom to which a hydroxyl group is bonded or to an atom two positions away from the atom to which a hydroxyl group is bonded). The number of hydroxyl groups, the number of acetal bonds, and the proportion of hydroxyl groups to be acetalized are the same as in the hydroxyl group-containing polymer having a skeleton other than the structure represented by Chemical Formula (2a).

In a case where the hydroxyl group-containing polymer has, for example, a branched structure represented by Chemical Formula (2b), that is, the polymer is a polymer in which the polymer A is grafted with the polymer B (such a polymer having a skeleton is referred to as “poly A-graft-poly B”), the hydroxyl group-containing polymer acetalized with the compound 1 can be represented by Chemical Formula (10b). The compound represented by Chemical Formula (2b) is an example of a hydroxyl group-containing polymer, and the compound represented by Chemical Formula (10b) is an example of a hydroxyl group-containing polymer acetalized by Chemical Formula 1.

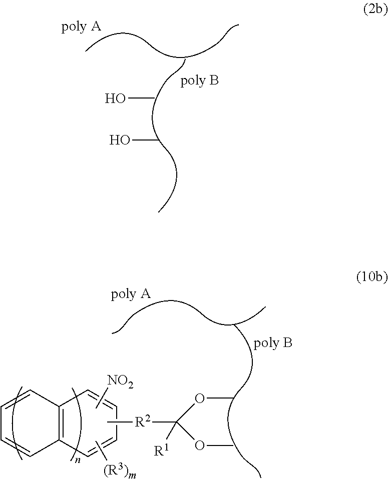

In the compound 10b, only the poly B which is a side chain (graft chain) is acetalized, but the poly A which is a main chain may also be acetalized in the main chain in addition to the side chain. In addition, in the poly B, it is preferable that two or more hydroxyl groups are present at positions adjacent to or separated by two atoms. Further, in a case where the poly A is acetalized, it is preferable that two or more hydroxyl groups are also present in the poly A at positions adjacent to or separated by two atoms.

In the compound 1, R¹ represents a hydrogen atom, a methyl group, or a cyclopropyl group, R¹ may represent a hydrogen atom or a methyl group, and R¹ may represent a hydrogen atom. R² represents a single bond, an alkylene group having 1 to 3 carbon atoms, an alkenylene group having 2 to 3 carbon atoms, or a group represented by —C(CR¹O)—, R² may represent a single bond, an alkylene group having 1 to 3 carbon atoms, or an alkenylene group having 2 to 3 carbon atoms, R² may represent a single bond or an alkylene group having 1 to 3 carbon atoms, and R² may represent a single bond.

In the compound 1, R³ each independently represents an alkyl group having 1 to 3 carbon atoms, a hydroxyalkyl group having 1 to 3 carbon atoms, a hydroxyl group, a halogen atom, an alkoxycarbonyl group, a nitro group, or an arylsulfonyl group. R³ may each independently represent an alkyl group having 1 to 3 carbon atoms or a hydroxyl group, where the alkyl group having 1 to 3 carbon atoms may be an ethyl group or a propyl group, or may be an ethyl group.

n in the compound 1 represents an integer of 0 or 1 representing a monocycle or a fused ring. n may represent 0. m represents the number of R³ bonded to an aromatic ring, and m represents an integer of 0 to 4 in a case where n represents 0, and m represents an integer of 0 to 6 in a case where n represents 1. m may represent an integer of 0 to 3 or an integer of 0 to 2 in any case where n represents 0 or 1, and it is preferable that m represents 0 or 1.

In the compound 1, the nitro group needs to be adjacent to a group represented by —R²—CR¹O or to R³ which represents an alkyl group having 1 to 3 carbon atoms. This is because it is preferable to cause a crosslinking reaction by light (ultraviolet rays), and the crosslinking occurs by a dimerization reaction or the like of the nitroso group generated by irradiation with light (ultraviolet rays).

As the compound 1, at least one of the compounds represented by the following formulae can be employed (in the formulae, X represents a halogen atom or a methyl group).

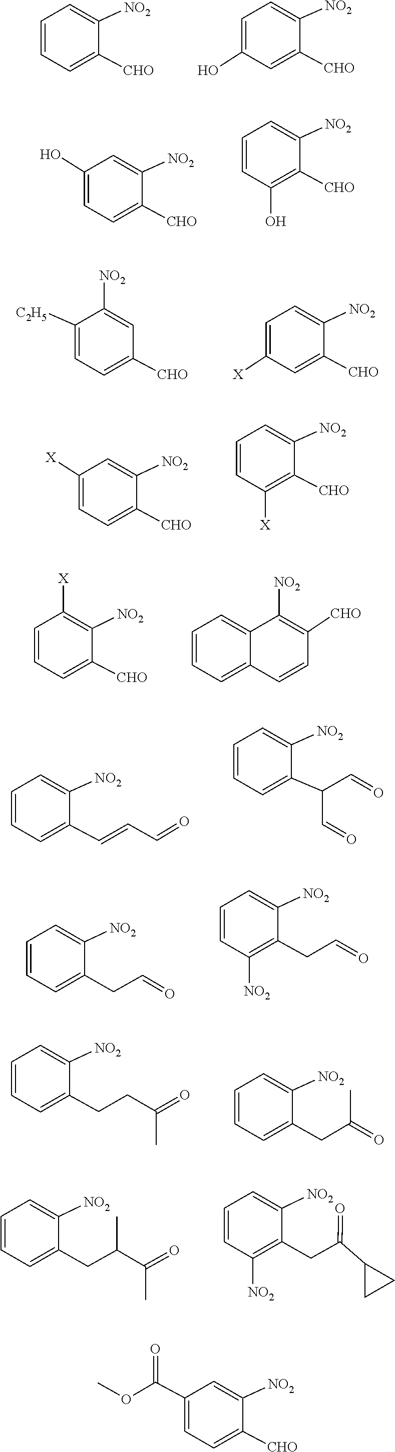

It is preferable that the compound 1 is at least one of the compounds represented by the following formulae.

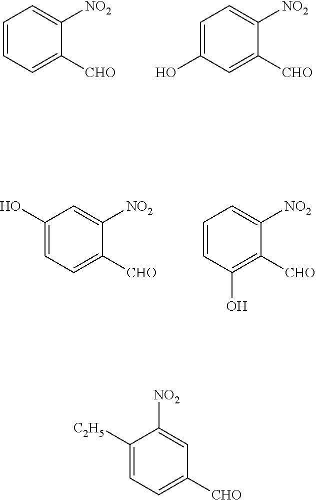 [SMILES: O=Cc1ccccc1[N+](=O)[O-] ; O=Cc1cc(O)ccc1[N+](=O)[O-] ; O=Cc1ccc(O)cc1[N+](=O)[O-] ; O=Cc1c(O)cccc1[N+](=O)[O-] ; CCc1ccc(C=O)cc1[N+](=O)[O-]]

At least one compound represented by the following formula is more preferable as the compound 1.

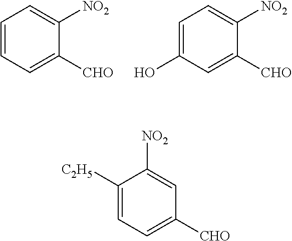 [SMILES: O=Cc1ccccc1[N+](=O)[O-] ; O=Cc1cc(O)ccc1[N+](=O)[O-] ; CCc1ccc(C=O)cc1[N+](=O)[O-]]

Among the polymer materials, suitable examples thereof include a polymer material containing a hydroxyl group-containing polymer which is acetalized with a compound represented by Formula (1a), in which the hydroxyl group-containing polymer contains two or more hydroxyl groups in a main chain and/or a side chain, the acetalization is based on a reaction between a group represented by —R²—CR¹O in General Formula (1a) and two hydroxyl groups in the hydroxyl group-containing polymer, and the polymer material forms a crosslinked substance by irradiation with light.

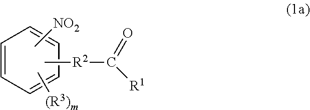

In the formula, R¹ represents a hydrogen atom, R² represents a single bond, an alkylene group having 1 to 3 carbon atoms, or an alkenylene group having 2 to 3 carbon atoms, R³ represents a hydroxyl group, a methyl group, or an ethyl group, and m represents an integer of 0 to 4. Here, the nitro group in the formula is adjacent to a group represented by —R²—CR¹O or is adjacent to R³ which represents a methyl group or an ethyl group. Further, it is preferable that R² represents a single bond, R³ represents a hydroxyl group or an ethyl group, and m represents an integer of 0 or 1.

The polymer material can be crosslinked even with ultraviolet rays (UVA) having a long wavelength of 320 nm or greater. For example, the crosslinking can be performed by irradiation with ultraviolet rays under conditions of a wavelength of 365 nm and an irradiation amount of 500 mJ/cm² or greater. The irradiation time can be set to, for example, 2 to 60 seconds or 5 to 30 seconds under irradiation conditions of a wavelength of 365 nm and an intensity of 250 mW/cm², and can be set to 20 seconds to 10 minutes or 1 to 5 minutes under irradiation conditions of a wavelength of 365 nm and an intensity of 25 mW/cm². In addition, the crosslinking reaction can also be carried out under a condition that irradiation with ultraviolet rays is performed in air (oxygen).

In a case where 2-nitrobenzaldehyde is used as the compound 1, an example of the crosslinking reaction is represented by the following chemical formula.

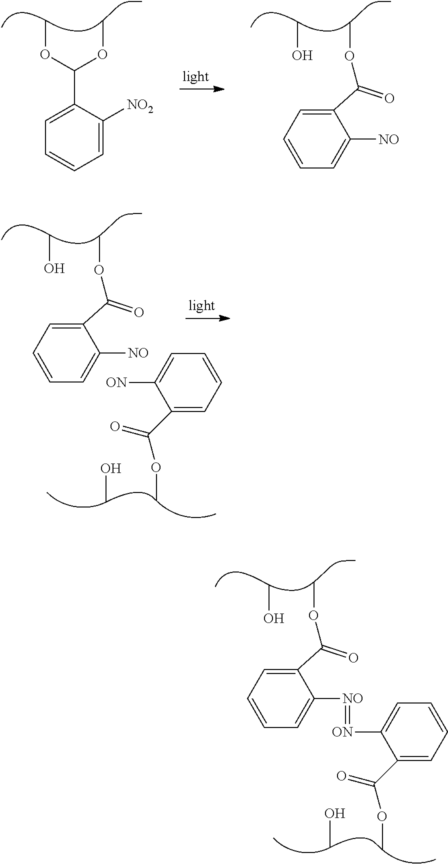

As is clear from the reaction formula shown above, in the polymer material according to the present invention, the low molecular weight component is not liberated even after the photoreaction, and addition of a low molecular weight compound that can be liberated after the crosslinking and is involved in the photoreaction is not required. In addition, the polymer material can be crosslinked in a state of a liquid phase containing a solvent. Examples of the solvent for preparing a liquid phase state include water, a water-miscible organic solvent (which denotes an organic solvent miscible with water), and a combination of one or more kinds of water-miscible organic solvents and water. Examples of the water-miscible organic solvents include polar solvents such as 1,1,1,3,3,3-hexafluoroisopropanol (HFIP) [HFIP is also used as a synthetic solvent for a polymer material], dimethylformamide (DMF), dimethylsulfoxide (DMSO), and an alcohol (preferably an alcohol having 1 to 6 carbon atoms and more preferably an alcohol having 1 to 3 carbon atoms, and examples thereof include methanol, ethanol, propanol, ethylene glycol, and propylene glycol). Examples of the combination (mixed solvent) of one or more kinds of water-miscible organic solvents and water include a water-alcohol mixed solvent. Various electrolytes can also be added to the solvent. Further, the water-miscible organic solvent includes an organic solvent that can be arbitrarily mixed with water (20° C.) and an organic solvent having a solubility of 5 g/L or greater in water (20° C.).

The hydroxyl group-containing polymer to be acetalized with the compound 1 is a polymer containing two or more hydroxyl groups in the main chain and/or a side chain, and may further have at least one of a carboxy group and a sulfonic acid group. The carboxy group and the sulfonic acid group may form salts such as an alkali metal salt such as sodium or potassium, an alkaline earth metal salt such as magnesium or calcium, and an ammonium salt. Examples of the hydroxyl group-containing polymer to be acetalized include at least one of the following polymers (a), (b), and (c), and a polymer having at least one of a carboxy group and a sulfonic acid group (the carboxy group and the sulfonic acid group may form a salt as described above) in this polymer.
(a) Polyvinyl alcohol
(b) At least one vinyl alcohol copolymer selected from a linear vinyl alcohol copolymer and a graft vinyl alcohol copolymer
(c) At least one glycerol polymer selected from linear polyglycerol, hyperbranched glycerol, glycerol dendron, and glycerol dendrimer

As for the polyvinyl alcohol (a) and the vinyl alcohol portion (b), polymers having a polymerization degree of 500 to 3500 and a saponification degree of 70% to 100% by mole can be used. Examples of the vinyl alcohol graft copolymer (b) include polyethylene glycol-graft-polyvinyl alcohol. Examples of the vinyl alcohol copolymer (b) include an ethylene vinyl alcohol copolymer and saponified product of an ethylene vinyl acetate copolymer.

Examples of the form in which the hydroxyl group-containing polymer contains a carboxy group or a sulfonic acid group (or a salt thereof) include a form in which a carboxy group or a sulfonic acid group (or a salt thereof) is directly bonded to the main chain and/or a side chain of the hydroxyl group-containing polymer and a form in which a hydroxyl group-containing polymer is acetalized with a compound having a carboxy group or a sulfonic acid group (or a salt thereof) and a formyl group (for example, 4-formylbenzoic acid or glyoxylic acid).

Hereinafter, some embodiments of the polymer material will be described. The polymer material according to a first embodiment is a polymer material containing polyvinyl alcohol acetalized with a compound represented by the following formula. This polymer material is insolubilized in a solvent by being irradiated with ultraviolet rays in a solid state and being brought into contact with water. In addition, a gel insoluble in water is generated by irradiating a solution with ultraviolet rays, the solution being in a state of a solvent containing a water-miscible organic solvent.

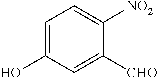 [SMILES: O=Cc1cc(O)ccc1[N+](=O)[O-]]

The polymer material according to a second embodiment is a polymer material containing polyethylene glycol-graft-polyvinyl alcohol which is acetalized with a compound represented by the following formula. This polymer material is insolubilized in water by being irradiated with ultraviolet rays in a solid state. In addition, a gel insoluble in water is generated by irradiating the solution with ultraviolet rays in a solution state of a solvent containing a water-miscible organic solvent.

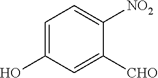 [SMILES: O=Cc1cc(O)ccc1[N+](=O)[O-]]

The polymer material according to a third embodiment is a polymer material containing polyethylene glycol-graft-polyvinyl alcohol which is acetalized with a compound represented by the following formula. This polymer material is insoluble in water by being irradiated with ultraviolet rays in a solid state and subsequently humidified. In addition, a gel insoluble in water is generated by irradiating the solution with ultraviolet rays in a solution state of a solvent containing a water-miscible organic solvent.

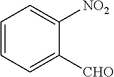 [SMILES: O=Cc1ccccc1[N+](=O)[O-]]

The polymer material according to a fourth embodiment is a polymer material containing polyvinyl alcohol acetalized with a compound represented by the following formula. This polymer material generates a gel insoluble in water by being irradiated with ultraviolet rays in the form of a solution of a solvent containing water and a water-miscible organic solvent (for example, an alcohol having 1 to 3 carbon atoms, such as methanol, ethanol, or propanol). In addition, the polymer material is insolubilized in a solvent containing water and/or a water-miscible organic solvent by being irradiated with ultraviolet rays in a solid state.

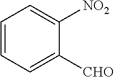 [SMILES: O=Cc1ccccc1[N+](=O)[O-]]

The polymer material according to a fifth embodiment is a polymer material that contains polyvinyl alcohol containing a carboxy group or a sulfonic acid group (the carboxy group and the sulfonic acid group may form a salt), which is acetalized with a compound represented by the following formula. This polymer material generates a gel by being irradiated with ultraviolet rays in a solution state of a solvent containing a solvent containing at least one of (preferably any one of these) water, a buffer solution, and a base forming a salt of a carboxy group or a sulfonic acid group. Further, examples of the base that forms a salt of a carboxy group or a sulfonic acid group include hydroxides of alkali metals or alkaline earth metals such as sodium hydroxide, potassium hydroxide, and calcium hydroxide (the same applies to the base hereinafter). In addition, examples of the buffer solution include a phosphate buffer solution, an acetate buffer solution, a citrate buffer solution, a citrate phosphate buffer solution, a borate buffer solution, and a tartrate buffer solution (the same applies to the buffer solution hereinafter).

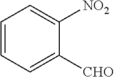 [SMILES: O=Cc1ccccc1[N+](=O)[O-]]

In the fifth embodiment, the polymer material composed of polyvinyl alcohol having a carboxy group or a sulfonic acid group (the carboxy group and the sulfonic acid group may form a salt) may be a polymer material composed of polyvinyl alcohol in which a carboxy group or a sulfonic acid group is directly bonded to the main chain of polyvinyl alcohol, or may be a polymer material composed of polyvinyl alcohol which is acetalized with a compound having a carboxy group or a sulfonic acid group and a formyl group.

The polymer material according to a sixth embodiment is a polymer material comprising polyvinyl alcohol having a carboxy group or a sulfonic acid group (the carboxy group and the sulfonic acid group may form a salt), which has been acetalized with a compound represented by the following formula. This polymer material generates a gel by being irradiated with ultraviolet rays in a state of being dissolved in an aqueous solution containing a base that forms a salt of a carboxy group or a sulfonic acid group.

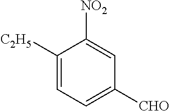 [SMILES: CCc1ccc(C=O)cc1[N+](=O)[O-]]

In the sixth embodiment, the polymer material composed of polyvinyl alcohol containing a carboxy group or a sulfonic acid group (the carboxy group and the sulfonic acid group may form a salt) may be a polymer material composed of polyvinyl alcohol in which a carboxy group or a sulfonic acid group is directly bonded to the main chain of polyvinyl alcohol, or may be a polymer material composed of polyvinyl alcohol which is acetalized with a compound containing a carboxy group or a sulfonic acid group and a formyl group.

Among the polymer materials according to the sixth embodiment, the polymer material containing polyvinyl alcohol, which is acetalized with a compound represented by the following formula and a compound containing a carboxy group or a sulfonic acid group and a formyl group, not only generates a gel by being irradiated with ultraviolet rays in a state of being dissolved in an aqueous solution containing a base that forms a salt of the carboxy group or the sulfonic acid group, but also has high usefulness since the gel stably maintains the state even after reaching equilibrium swelling by being immersed in water. Further, the compound containing a carboxy group or a sulfonic acid group and a formyl group is preferably a compound containing a formyl group and a carboxy group (for example, 4-formylbenzoic acid or glyoxylic acid).

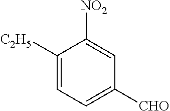 [SMILES: CCc1ccc(C=O)cc1[N+](=O)[O-]]

The aqueous solution of the polymer material according to the sixth embodiment may further contain cultured cells and a liquid culture medium for cell culture. There is no limitation on the type of cultured cells, and examples include human liver cancer-derived cells (HepG2 cells) or HeLa cells.

Examples of the liquid culture medium for cell culture include Dulbecco's Modified Eagle's Medium (DMEM medium), McCoy's 5A Medium, RPMI 1640 medium, Glasgow's MEM (GMEM), Ham's F-12 medium, and α-MEM medium. The DMEM medium contains inorganic salts such as calcium chloride, ferric nitrate, magnesium sulfate, potassium chloride, sodium chloride, and sodium dihydrogen phosphate, vitamins such as choline chloride, calcium pantothenate, folic acid, mositol, nicotinamide, pyridoxal hydrochloride, riboflavin, and thiamine hydrochloride, amino acids such as glycine, L-arginine, L-cystine, L-histidine, L-isoleucine, L-leucine, L-lysine, L-methionine, L-phenylalanine, L-serine, L-threonine, L-tryptophan, L-tyrosine, and L-valine, and other components such as D-glucose or dextrose, pyruvic acid, and sodium hydrogen carbonate. Further, the liquid culture medium for cell culture may contain a growth supplement such as fetal bovine serum (FBS).

The shape of the photocrosslinked gel of the polymer material according to the sixth embodiment can be largely deformed without being destroyed even in a case of being pressurized. For example, even in a case where a load of about 3,000 g is applied to a gel test piece having a cylindrical shape (φ6 mm×1.5 mm), the relative cross-sectional area (cross-sectional area/original cross-sectional area) reaches about 7 without the test piece being broken. In a case where the stress-strain properties of the photocrosslinked gel of the polymer material according to the sixth embodiment are measured, the elastic modulus is small so that the material is supple in a range where the compressive strain is small, and a high elastic force is exhibited in a case where the compressive strain is increased. In addition, in a case where a solution of the polymer material according to the sixth embodiment is introduced between the adherends (it is preferable that at least one of the adherends transmits ultraviolet rays) and irradiated with ultraviolet rays to form a photocrosslinked gel, the adherends can firmly adhere to each other. The photocrosslinked gel of the solution of the polymer material according to the sixth embodiment can be swollen with water or the like, and the water-swollen substance exhibits sufficient mechanical strength for handling by hand even in a state where the equilibrium swelling degree is high and the water content is greater than 99.9%.

The above-described polymer material can be used as a liquid material in which the polymer material is dissolved, dispersed, or swollen. The polymer material can be used as a photolithography material, and in this case, the above-described liquid material can be spin-coated or the like to form a thin film. In addition, a crosslinked substance can be formed by irradiating the polymer material with ultraviolet rays, but even in a case of ultraviolet rays (UVA) having a long wavelength of 320 nm or greater, crosslinking can be performed, and addition of a low molecular weight compound that can be liberated after crosslinking and is involved in a photoreaction is not required, and the low molecular weight component can be crosslinked in a state of a liquid phase containing an aqueous solvent without being liberated even after the photoreaction.

The polymer material according to the first to fourth embodiments can be used as a cell adhesion inhibitor by being irradiated with ultraviolet rays to form a crosslinked substance. In addition, the polymer material according to the fifth and sixth embodiments can be used as a cell culture gel by being irradiated with ultraviolet rays to form a crosslinked substance. In particular, cells can be cultured in the gel by allowing the aqueous solution to contain the cultured cells and the liquid culture medium for cell culture.

### EXAMPLES

Hereinafter, the present invention will be described with reference to examples, but the present invention is not limited to the following examples.

### (Example 1) Synthesis 1 of OHNBA-PVA

5-Hydroxy-2-nitrobenzaldehyde (OHNBA)-modified polyvinyl alcohol (OHNBA-PVA) was synthesized as follows. That is, PVA (polymerization degree: 1,500, saponification degree: 86% to 90% by mole) and OHNBA were dissolved in 1,1,1,3,3,3-hexafluoroisopropanol (HFIP) such that the concentrations thereof respectively reached 4.4% by weight and 0.38% by weight, a 12 N HCl aqueous solution was added thereto such that the concentration thereof reached 8.4 mmol/kg, and the solution was stirred at 25° C. for 3 days to promote an acetal addition reaction. Thereafter, the mixture was recrystallized from 1,2-dichloroethane, washed with ethanol (EtOH), and dried at 60° C., thereby obtaining a target polymer (OHNBA-PVA). The amount of OHNBA charged relative to PVA was 4.1% by weight.

### (Example 2) Synthesis 2 of OHNBA-PVA

OHNBA-PVA was synthesized in the same manner as in Example 2 except that the amount of OHNBA charged to PVA was set to 3.2% by weight.

### [Photoresponse 1 of OHNBA-PVA]

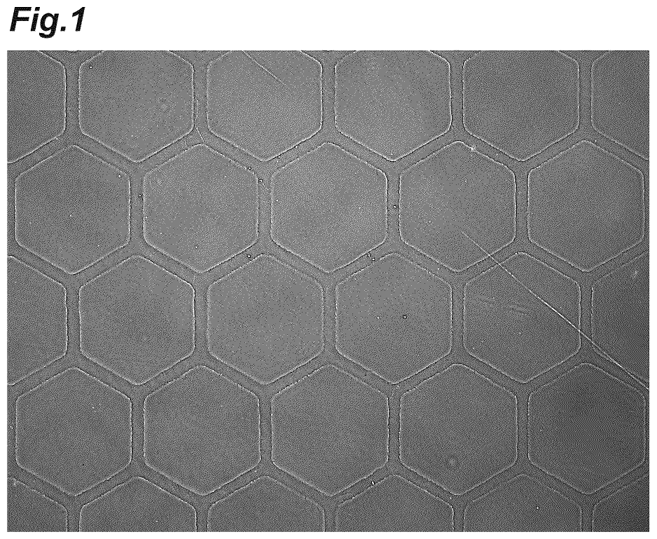

OHNBA-PVA obtained in Example 1 was insoluble in water in the non-irradiated state, but was dissolved in 70% ethanol (30% water). In a case where a polystyrene dish was spin-coated with the HFIP solution of OHNBA-PVA of Example 1 with an OHNBA charging ratio of 4.1% by weight to form a polymer thin layer, and the polymer thin layer was irradiated with light for 20 s along a honeycomb pattern under the conditions of a wavelength of 365 nm and an intensity of 64 mW/cm², immersed in water once, and washed with 70% ethanol, it was confirmed that only the irradiation region remained (FIG. 1). However, in a case where it was washed with 70% ethanol without being immersed in water, OHNBA-PVA was dissolved regardless of the irradiation and removed from the surface of the polystyrene dish. Further, it was confirmed that the region where OHNBA-PVA remained had cell adhesion inhibitory properties.

FIG. 1 shows a state of an OHNBA-PVA coating layer (OHNBA-PVA remained along the irradiation region) after OHNBA-PVA of Example 1 was pattern-irradiated (on a honeycomb line, one side: 500 μm) at 365 nm with an intensity of 64 mW/cm² for 20 s, immersed in water once, and washed with 70% ethanol.

### [Photoresponse 2 of OHNBA-PVA]

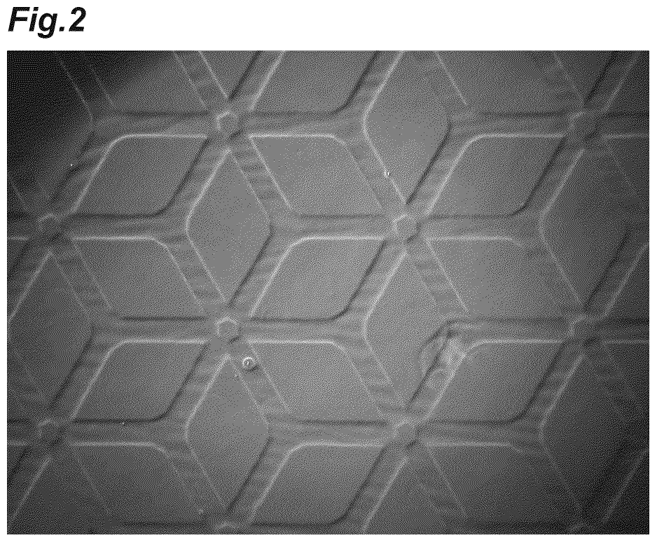

OHNBA-PVA obtained in Example 2 was insoluble in water in a state of not being irradiated with light, but was dissolved in 70% ethanol. A polystyrene dish was spin-coated with the HFIP solution of OHNBA-PVA of Example 2 with an OHNBA charging introduction rate of 3.2% by weight to form a polymer thin layer. It was confirmed that the polymer thin layer was removed by being washed with water under a condition of not being irradiated, but swollen in water to form a gel layer in a case where the polymer thin layer was irradiated with ultraviolet rays having a wavelength of 365 nm and an intensity of 280 mW/cm² for 15 s and water was introduced thereto (FIG. 2). Further, it was confirmed that the region where OHNBA-PVA remained had cell adhesion inhibitory properties.

FIG. 2 shows a state of an OHNBA-PVA coating layer (OHNBA-PVA remained along the irradiation region and was swollen) after OHNBA-PVA of Example 2 was pattern-irradiated at 365 nm with 280 mW/cm² for 15 s and immersed in water.

### (Example 3) Synthesis 1 of OHNBA-PVAPEG

A 5-hydroxy-2-nitrobenzaldehyde (OHNBA)-modified polyethylene glycol-graft-polyvinyl alcohol copolymer (OHNBA-PVAPEG) was synthesized as follows. That is, polyethylene glycol-graft-polyvinyl alcohol (Kollicoat IR) and OHNBA were dissolved in HFIP such that the concentration thereof was 3.5% by weight, a 12 N HCl aqueous solution was added thereto such that the concentration thereof was 8.0 mmol/kg, and the solution was stirred at 15° C. for 3 days to promote an acetal addition reaction. Thereafter, the mixture was recrystallized from 1,2-dichloroethane, washed with ethanol, and dried at 60° C., thereby obtaining a target polymer.

### (Example 4) Synthesis 2 of OHNBA-PVAPEG

OHNBA-PVAPEG was synthesized in the same manner as in Example 3 except that the OHNBA was dissolved in HFIP such that the concentration thereof reached 0.32% by weight.

### [Photoresponse of OHNBA-PVAPEG]

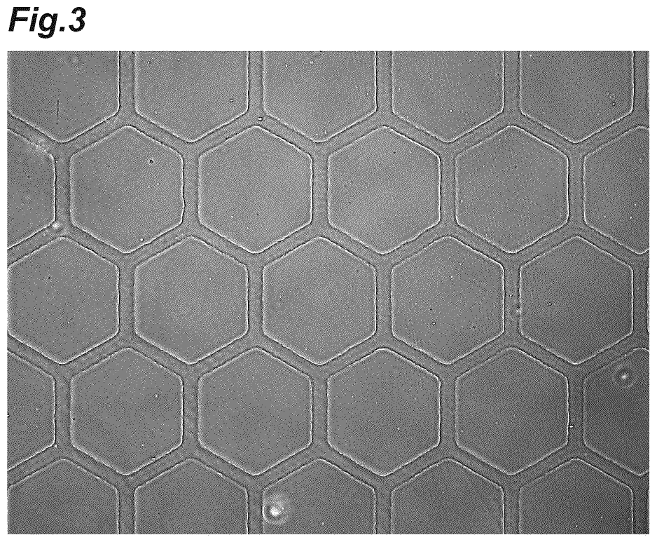

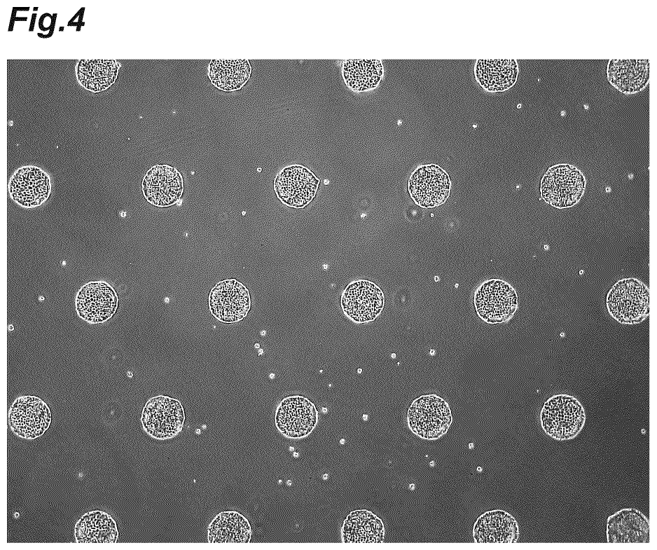

In a case where a polystyrene dish was spin-coated with the HFIP solution of OHNBA-PVAPEG obtained in Example 3 to form a polymer thin layer, and the polymer thin layer was irradiated with light for 15 s along a honeycomb pattern under the conditions of a wavelength of 365 nm and an intensity of 280 mW/cm², and washed with water, it was observed that the entire polymer was removed from the non-irradiation region while the polymer remained in the irradiation region (FIG. 3). Further, it was confirmed that the region in which OHNBA-PVAPEG remained had cell adhesion inhibitory properties (FIG. 4).

FIG. 3 shows a state of the OHNBA-PVAPEG coating layer (OHNBA-PVAPEG remained along the irradiation region) after the layer was pattern-irradiated (on a honeycomb line, one side: 500 μm) at 365 nm with an intensity of 280 mW/cm² for 15 s and washed with water. FIG. 4 shows cell patterning (cell adhesion in the non-irradiated region) formed by pattern-irradiating (inverted polka dots, distance between centers of dots: 900 μm) the OHNBA-PVAPEG coating layer at 365 nm with an intensity of 120 mW/cm² for 20 s, washing the coating layer with water, and seeding Madin-Darby Canine Kidney (MDCK) cells.

### (Example 5) Synthesis 1 of NBA-PVAPEG

A 2-nitrobenzaldehyde-modified polyethylene glycol-graft-polyvinyl alcohol copolymer (NBA-PVAPEG) was synthesized as follows. That is, polyethylene glycol-graft-polyvinyl alcohol (Kollicoat IR) was dissolved in HFIP so that the concentration of 2-nitrobenzaldehyde reached 7.4% by weight, a 12 N HCl aqueous solution was added thereto so that the concentration thereof reached 14 mmol/kg, and the solution was stirred at 25° C. for 3 days to promote the acetal addition reaction. Thereafter, the product was recrystallized with 1,2-dichloroethane, washed with ethanol, and dried at 40° C., thereby obtaining a target polymer.

### (Example 6) Synthesis 2 of NBA-PVAPEG

A polymer was obtained in the same manner as in Example 5 except that 2-nitrobenzaldehyde was dissolved in HFIP such that the concentration thereof reached 0.52% by weight.

### [Photoresponse of NBA-PVAPEG]

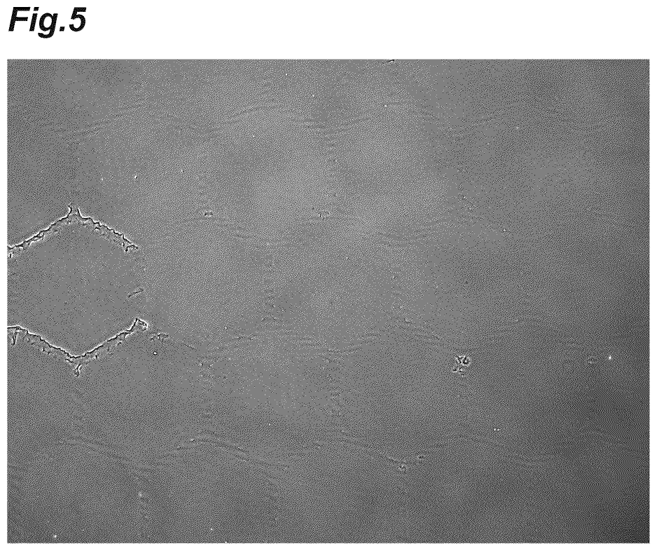

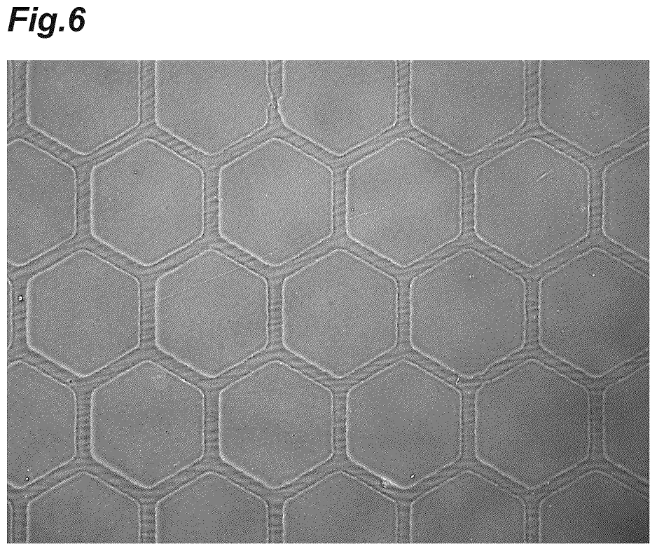

In a case where a polystyrene dish was spin-coated with the HFIP solution of NBA-PVAPEG obtained in Example 5 to form a polymer thin layer, and the polymer thin layer was irradiated with light for 30 s along a honeycomb pattern under the conditions of a wavelength of 365 nm and an intensity of 280 mW/cm², and washed with water, it was confirmed that the entire polymer was removed from the non-irradiation region and almost the entire polymer was removed in the irradiation region (FIG. 5). However, it was observed that in a case where the polymer was placed under a high-humidity condition (cell culture incubator) for about 10 seconds before washing and washed with water, the polymer was dissolved and removed in the non-irradiation region while the polymer remained in the irradiation region (FIG. 6). In addition, it was confirmed that the region in which NBA-PVAPEG remained had cell adhesion inhibitory properties.

FIG. 5 shows a state after the NBA-PVAPEG coating layer was pattern-irradiated at 365 nm with an intensity of 280 mW/cm² for 30 s (on a honeycomb line, one side: 500 μm) and washed with water. FIG. 6 shows a state after the NBA-PVAPEG coating layer was pattern-irradiated at 365 nm, with an intensity of 280 mW/cm² for 30 s (on a honeycomb line, one side: 500 μm), placed in a high-humidity environment for 10 seconds, and washed with water.

### (Example 7) Synthesis 1 of NBA-PVA

2-Nitrobenzaldehyde-modified polyvinyl alcohol (NBA-PVA) was synthesized as follows. That is, polyvinyl alcohol having an average polymerization degree of about 1,500 and a saponification degree of about 88% by mole and 2-nitrobenzaldehyde were dissolved in HFIP such that the concentration thereof reached 7.7% by weight, a 12 N HCl aqueous solution was added thereto such that the concentration thereof reached 9.1 mmol/kg, and the solution was stirred at 25° C. for 2 days to carry out an acetal addition reaction. Thereafter, the mixture was recrystallized with toluene, washed with ethyl acetate, and dried at 40° C., thereby obtaining a target polymer.

### (Example 8) Synthesis 2 of NBA-PVA

A polymer was obtained in the same manner as in Example 6 except that 2-nitrobenzaldehyde was dissolved in HFIP such that the concentration thereof reached 0.3% by weight.

### [Photoresponsive Sol-Gel Transition of NBA-PVA Solution]

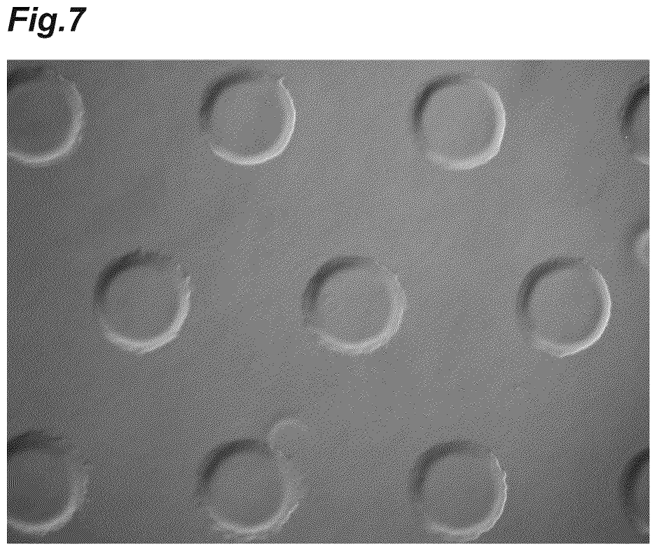

In a case where the solid of NBA-PVA obtained in Example 7 was dissolved in a mixed solvent of water and ethanol at a mixing ratio of 1:1 such that the concentration thereof reached 10% by weight, a one-phase colorless and transparent solution was obtained. In a case where a polystyrene surface was coated with the solution and uniformly irradiated with light for 5 seconds under the conditions of a wavelength of 365 nm and an intensity of 280 mW/cm² in a solution state, the solution state was transitioned to a firm gel state. In addition, in a case where the gel was irradiated with light through a photomask having a polka dot pattern (diameter: 0.15 mm, distance between centers: 0.9 mm) under the same conditions as described above and washed with water, it was observed that the gel was dissolved and removed in the non-irradiation region while the gel remained in a dot shape in the irradiation region, and the gel had strength not to easily collapse even in a case of being touched with a finger (FIG. 7).

FIG. 7 shows a gel remaining after a 10 wt % NBA-PVA water-ethanol solution was loaded, pattern-irradiated (polka dots, distance between centers: 900 μm) at 365 nm with an intensity of 280 mW/cm² for 5 s in a solution state, and washed with water.

### (Example 9) Synthesis of NBA-PVA-COOH

2-Nitrobenzaldehyde-modified partially carboxylated polyvinyl alcohol (NBA-PVA-COOH) was synthesized as follows. That is, in order to make the modified polymer water-soluble, the partially carboxylated polyvinyl alcohol (GOHSENX T-330) and 2-nitrobenzaldehyde were each heated and dissolved in HFIP such that the concentrations thereof respectively reached 6% by weight and 0.27% by weight, a 12 N HC aqueous solution was added thereto such that the concentration thereof reached 54 mmol/kg, and the solution was stirred at 25° C. for 3 days to promote an acetal addition reaction. After neutralization with sodium hydroxide, the neutralized solution was gradually put into stirring ethanol, and the precipitate was dried at 40° C., thereby obtaining a target polymer (NBA-PVA-COOH). As a result of measuring the UV absorbance of the polymer aqueous solution, the introduction rate estimated from the molar absorption coefficient of 2-nitrobenzaldehyde acetalized with glycerin (2-(2-nitrophenyl)-1,3-dioxan-5-ol), adduct analog to PVA) was almost the same as that estimated from the charging ratio.

### (Example 10) Synthesis of NBA-PVA-SO₃M

A polymer was obtained in the same manner as in Example 9 except that polyvinyl alcohol (GOHSENX L-3266) modified with a sulfonic acid group was used in place of the partially carboxylated polyvinyl alcohol (GOHSENX T-330). In a case where the introduction rate estimated from the molar absorption coefficient was determined in the same manner as in Example 9, it was confirmed that 2-nitrobenzaldehyde could be introduced into the polymer at the same proportion as in Example 9.

### [Photoresponsive Sol-Gel Transition of NBA-PVA-COOH Aqueous Solution]

The solid of NBA-PVA-COOH obtained in Example 9 was put into water, heated, and dissolved, thereby obtaining a 5.9% one-phase colorless and transparent aqueous solution. When a polystyrene surface was coated with the solution and, while in the solution state, irradiated with light for 4 seconds under the conditions of a wavelength of 365 nm and an intensity of 280 mW/cm², the solution transitioned to an extremely firm gel state. In a case where 0.02 mL of this solution was added dropwise onto the polystyrene surface, a glass bottle having a diameter of 12 mm was placed thereon, and the glass bottle was irradiated with light for 4 seconds under the conditions of a wavelength of 365 nm and an intensity of 280 mW/cm², the glass bottle was adhered to the polystyrene surface. It was confirmed that a lid with a hook was attached to the bottle, the bottle was turned upside down, and a heavy object having a weight of about 300 g was hung from the hook in a state where the polystyrene was supported. In addition, it was confirmed that even in a case where a phosphate buffered aqueous solution having a pH of 7.4 was used in place of water, a 5.9% one-phase colorless transparent solution could be obtained, the same photoresponsive sol-gel transition was exhibited, and this gel did not exhibit toxicity to cultured cells.

### [Photoresponsive Sol-Gel Transition of NBA-PVA-SO₃M Aqueous Solution]

The photoresponsive sol-gel transition was found even in the aqueous solution of the polymer obtained in Example 10, in which 2-nitrobenzaldehyde was introduced into polyvinyl alcohol (GOHSENX K-434) modified with a sulfonic acid group. The elastic modulus of the gel was not as high as that of the aqueous solution of NBA-PVA-COOH having the same concentration.

### (Example 11) Synthesis of NBA-FBA-PVA

Polyvinyl alcohol modified with 2-nitrobenzaldehyde and 4-formylbenzoic acid (NBA-FBA-PVA) was synthesized as follows. That is, 0.13 g of polyvinyl alcohol (Mowiol 56-98, weight-average molecular weight: 195,000, saponification degree: 98% by mole) was dissolved in 4.9 g of HFIP, 0.057 g of 4-formylbenzoic acid was added thereto, and the solution was stirred until a uniform solution was formed, thereby obtaining a milky solution. The total amount of a solution obtained by dissolving 6.9 mg of a 12 N HCl aqueous solution and 7 mg of 2-nitrobenzaldehyde in 0.1 g of 2,2,2-trifluoroethanol (TFE) was put into the above-described milky solution, and the solution was stirred at 25° C. for 7 hours to promote an acetal addition reaction. After neutralization with sodium hydroxide, ethanol was added thereto, the solution was stirred, subjected to ultrasonic irradiation, a series of purification processes including removal of the supernatant were repeated twice, and the precipitate was dried at 80° C., thereby obtaining a target polymer (NBA-FBA-PVA).

### [Photo Sol-Gel Transition of NBA-FBA-PVA Aqueous Solution]

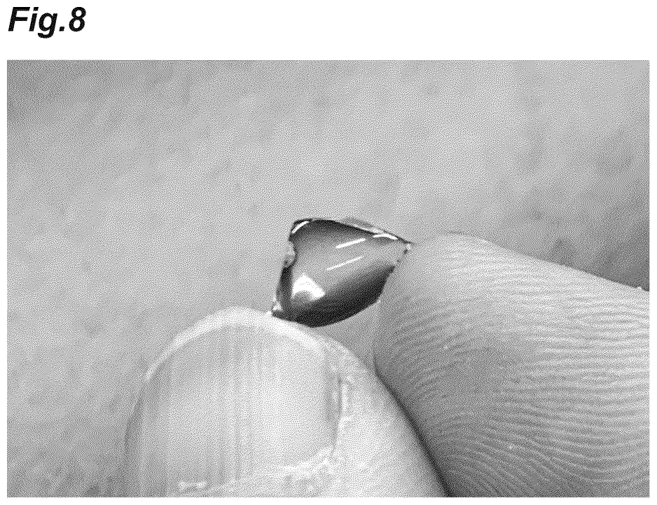

The solid of NBA-FBA-PVA obtained in Example 11 was dissolved in a sodium hydroxide aqueous solution in an amount equivalent to 4-formylbenzoic acid, which was estimated from the charging ratio, thereby obtaining a 4.7 wt % one-phase colorless and transparent aqueous solution. In a case where this solution was poured into a silicone resin mold to have a thickness of about 4 mm, and irradiated with light for 2 minutes under the conditions of a wavelength of 365 nm and an intensity of 40 mW/cm², the solution was transitioned to a gel state, and the gel could be taken out from the mold and picked up by hand (FIG. 8).

### (Example 12) Synthesis of ENBA-GoA-PVA

Polyvinyl alcohol modified with 4-ethyl-3-nitrobenzaldehyde and glyoxylic acid was synthesized as follows. That is, 67 mg of polyvinyl alcohol (Mowiol 56-98, weight-average molecular weight: 195,000, saponification degree: 98% by mole) was dissolved in 2.9 g of HFIP. A solution obtained by dissolving 4.6 mg of 4-ethyl-3-nitrobenzaldehyde in 0.2 g of a 5 wt % TFE solution of glyoxylic acid in another container was added thereto, and the solution was stirred. 0.4 g of a solution prepared by adding a TFE to a 12 N HCl aqueous solution such that the concentration thereof reached 0.1 mmol/g and 0.6 g of HFIP were mixed in another container and sufficiently stirred, and the total amount thereof was added to the reaction solution prepared in advance, and the solution was stirred at 70° C. for 1.5 hours to promote an acetal addition reaction. After neutralization with sodium hydroxide, ethanol was added thereto, the solution was stirred, and a series of purification processes of removing the supernatant was repeated twice to obtain a precipitate, and the precipitate was dried at 80° C., thereby obtaining a target polymer (ENBA-GoA-PVA).

### [Photo Sol-Gel Transition of ENBA-GoA-PVA Aqueous Solution]

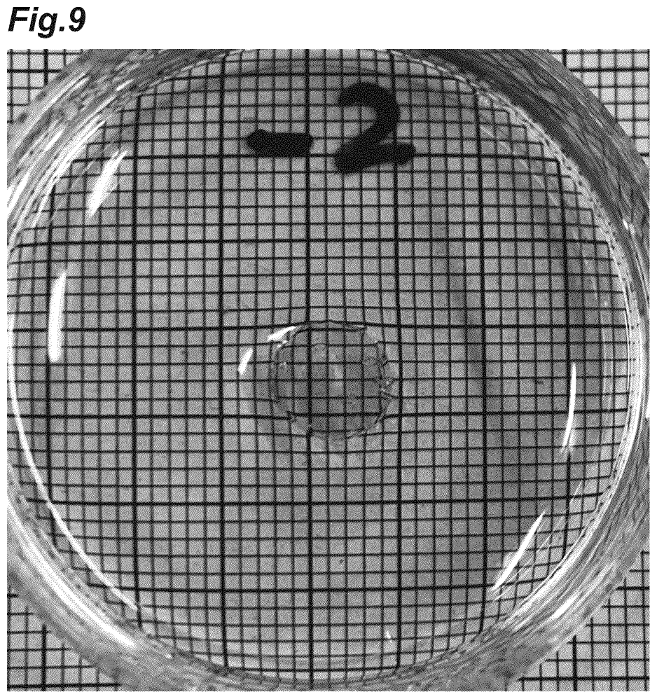

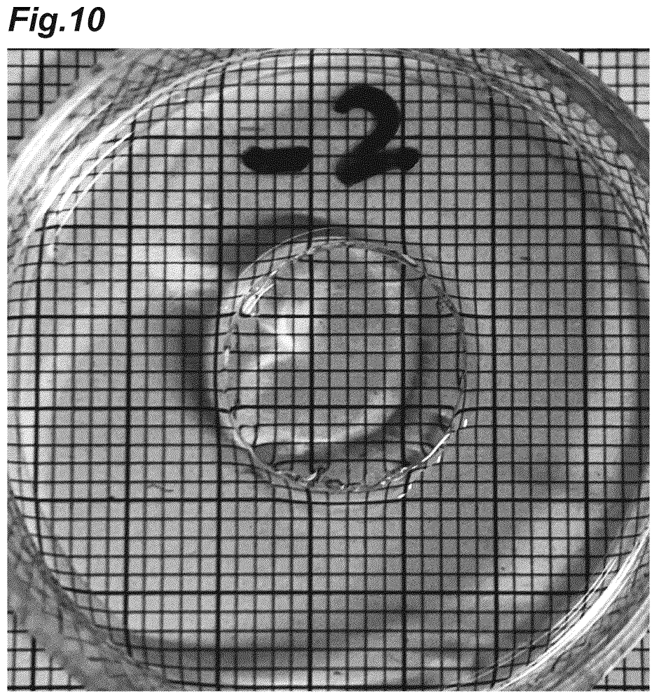

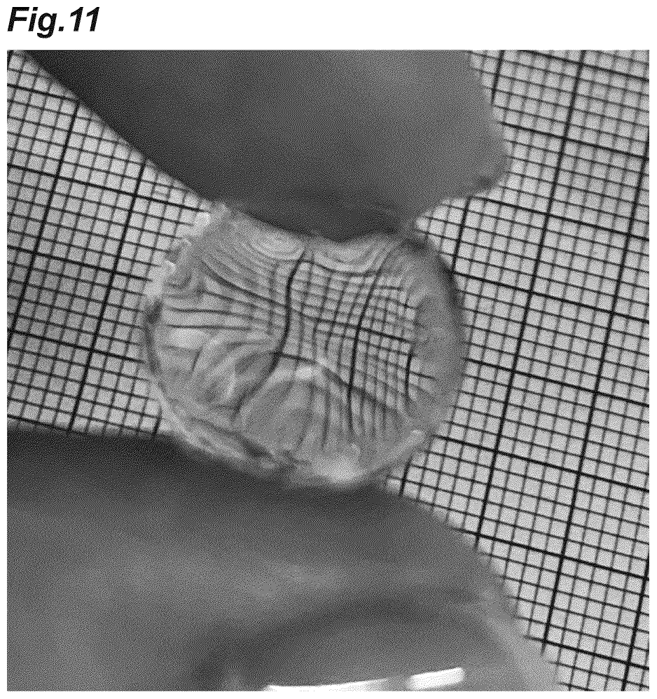

The solid of ENBA-GoA-PVA obtained under the same preparation conditions as in Example 12 was dissolved in a sodium hydroxide aqueous solution in an amount equivalent to glyoxylic acid, which was estimated from the charging ratio, thereby obtaining a 2 wt % one-phase colorless and transparent aqueous solution. This solution was poured into a cylindrical (φ6 mm×2 mm) silicone resin mold, and was irradiated with light for 2 minutes under the conditions of a wavelength of 365 nm and an intensity of 45 mW/cm², and thus the solution state was transitioned to a gel state, and the gel could be taken out from the mold and taken in hand (FIG. 9). In a case where this gel was immersed in water, the gel was gradually swollen and finally became a gel having a shape similar to the original gel, which was about twice (about 10 times in volume) the original dimensions of the original gel (FIG. 10). It was confirmed that even after the gel was swollen as described above and the water content reached about 99.8% (after equilibrium swelling), the gel (0.6 g) maintained strength sufficient to be handled by hand (FIG. 11).

### [Cell Culture in Photocrosslinked ENBA-GoA-PVA Gel]

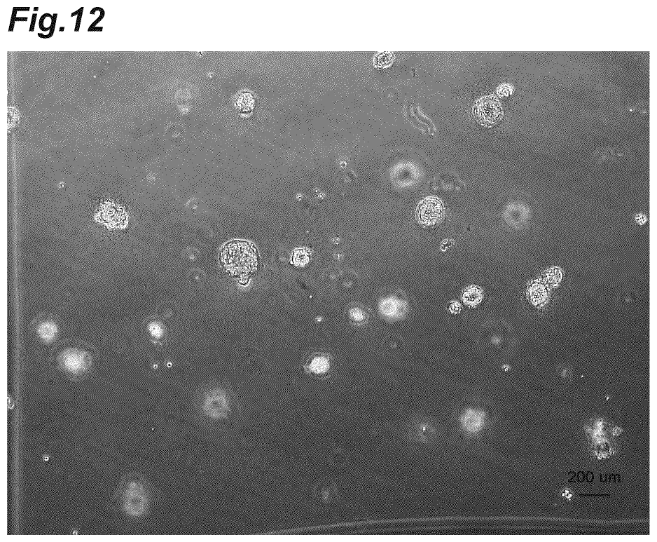

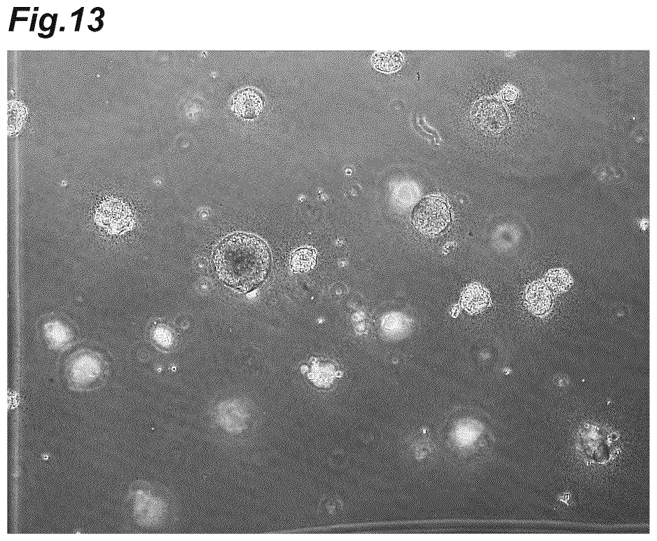

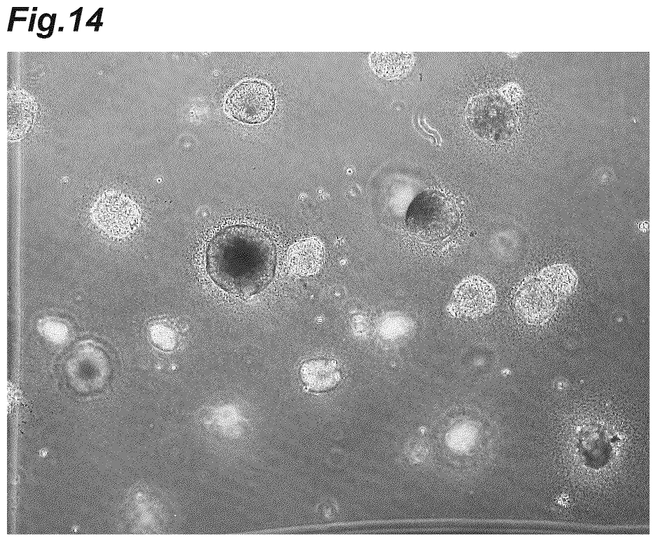

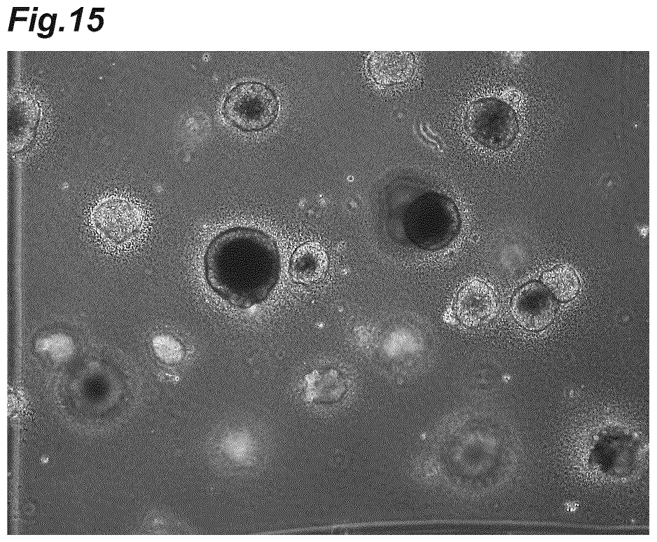

The solid of ENBA-GoA-PVA obtained in Example 12 was dissolved in a 1 N sodium hydroxide aqueous solution in an equivalent amount of glyoxylic acid estimated from the charging ratio and a phosphate buffer aqueous solution having a pH of 7.4, thereby obtaining a 2 wt % one-phase colorless and transparent aqueous solution. The solution was diluted to fourfold with Dulbecco's Modified Eagle's Medium (DMEM) containing 10% fetal bovine serum (FBS), and 0.1 mL of the solution to which a small amount of HeLa cells were added and dispersed therein was added dropwise to a polystyrene dish having a diameter of φ35 mm and immediately irradiated with light for 1 minute or 2 minutes under conditions of a wavelength of 365 nm and an intensity of 22 mW/cm² to form a gel. Dulbecco's Modified Eagle's Medium (DMEM) containing 10% fetal bovine serum (FBS) was loaded into the dish, and the state of the cells was observed while the culture medium was appropriately replaced. In the gel, the HeLa cells maintained a viable state, proliferated, and grew into spheroids with a diameter of greater than 200 μm in 2 to 3 weeks (FIGS. 12, 13, 14, and 15). During this period, the gel stably maintained the mechanical strength, and the three-dimensional position of the spheroids was constantly maintained at the same position. In this manner, it was confirmed that the photocrosslinked gel can permit the permeation of oxygen, carbon dioxide, sugar, and amino acids, and a sufficient amount of the components necessary for cell proliferation can be supplied from the culture medium.

FIGS. 12, 13, 14, and 15 show HepG2 cells maintained in a dispersed state by the photocrosslinking of a 0.5 wt % solution of ENBA-GoA-PVA, and the cells are respectively 13 days, 16 days, 19 days, and 22 days after the start of culture.

### (Example 13) Synthesis of ENBA-GoA-PVA

78 mg of polyvinyl alcohol (Mowiol 56-98, weight-average molecular weight: 195,000, saponification degree: 98 mol %) was dissolved in 2.6 g of HIP. A solution obtained by dissolving 3.2 mg of 4-ethyl-3-nitrobenzaldehyde in 80 mg of a 10 wt % TFE solution of glyoxylic acid was added to another container and the mixture was stirred. 0.4 g of a solution prepared by adding a TFE solution to a 12 N HCl aqueous solution such that the concentration thereof reached 0.1 mmol/g and 0.62 g of HFIP were mixed in another container, the mixture was sufficiently stirred, and the total amount of the mixture was put into the reaction solution prepared in advance, and the solution was stirred at 70° C. for 1 hour to promote an acetal addition reaction. After neutralization with NaOH, ethyl acetate was added thereto, the solution was stirred, and a series of purification processes of removing the supernatant was repeated twice to obtain a precipitate, and the precipitate was dried at 80° C., thereby obtaining a target polymer (ENBA-GoA-PVA).

### [Mechanical Properties of ENBA-GoA-PVA Photocrosslinked Gel]

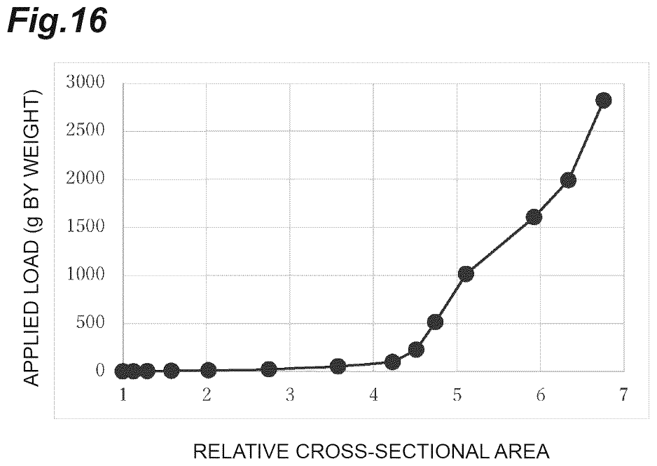

0.37 g of 0.2 N NaOH was added to 29 mg of ENBA-GoA-PVA of Example 13, water was further added thereto, and the solution was stirred to obtain a 2% ENBA-GoA-PVA aqueous solution, and the solution was diluted to two times with water to obtain a 1% aqueous solution. This solution was photocrosslinked in the same manner as in Example 12, and a gel test piece having a cylindrical shape (φ6 mm×1.5 mm) was obtained. The elastic modulus was measured by sandwiching the test piece between parallel glass plates, gradually increasing a load thereto, and measuring the cross-sectional area of the test piece while measuring the applied load. In this case, the load was applied until the applied load exceeded 3,000 g by weight, the load was gradually decreased to 0 g by weight, and this operation was repeated once more. In the first load application, the test piece reached a relative cross-sectional area (cross-sectional area/original cross-sectional area) of about 7 without being broken under a load of about 3000 g by weight (FIG. 16).

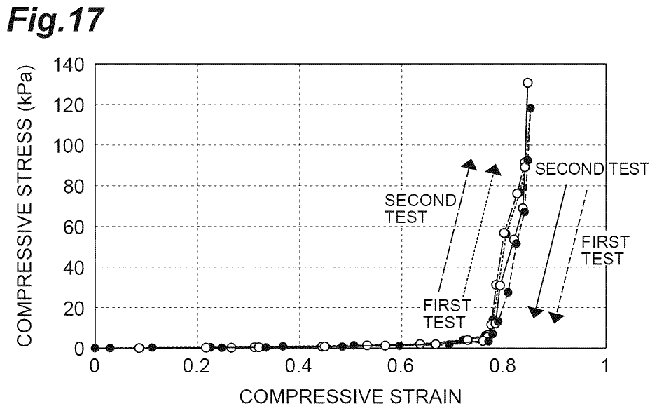

Based on the incompressibility (constant volume) of the gel, the compressive strain was calculated as 1-1/(relative cross-sectional area), and the stress-strain properties were analyzed by further calculating the compressive stress as (applied load)/(cross-sectional area). The compressive elastic modulus was about 2 kPa so that the test piece was flexible in a range where the compressive strain was small (<0.2), whereas the compressive stress rapidly increased at a compressive strain of 0.78, and high elasticity was exhibited. In addition, it was shown that the small test piece followed almost the same path in the first and second load applications and had high strength to withstand an applied load of 3000 g by weight (FIG. 17).

### [Adhesion Properties of ENBA-GoA-PVA Photocrosslinked Gel]

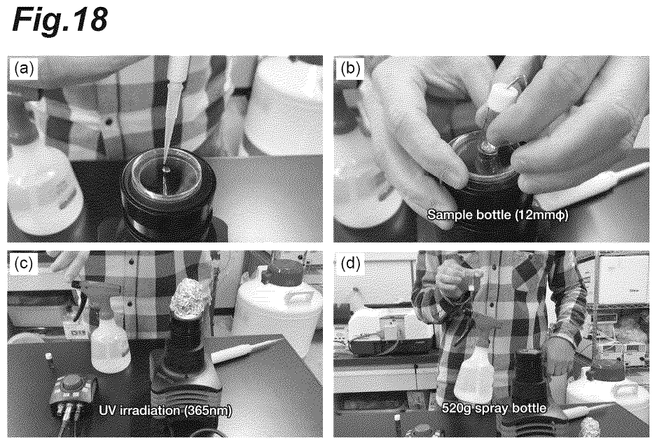

In order to verify that the gel generated by irradiation with light from the ENBA-GoA-PVA diluted solution had a strong adhesive force, an experiment was performed in the following manner (FIG. 18). One drop of a 2 wt % solution of ENBA-GoA-PVA obtained in Example 13 was dropped onto a lid of a culture dish (FIG. 18(a)), a screw tube bottle (Maruemu No. 02) having an outer diameter of 12 mm, in which a small amount of polyethyleneimine was immobilized on a bottom surface, was placed thereon with a hook attached to the opening (FIG. 18(b)), and the solution was irradiated with light having a wavelength of 365 nm (FIG. 18(c)) to promote a crosslinking reaction of the polymer. Thereafter, the lid of the culture dish was held and turned over, and the spray bottle having a weight of 520 g was hung on the hook attached to the opening of the screw tube bottle and lifted, and it was confirmed that the spray bottle was suspended (FIG. 18(d)). As a result, it was verified that the gel immediately formed by the photocrosslinking of the polymer aqueous solution containing 98% of water had mechanical properties of withstanding a high tensile stress.

### [Swelling Properties of ENBA-GoA-PVA Photocrosslinked Gel]

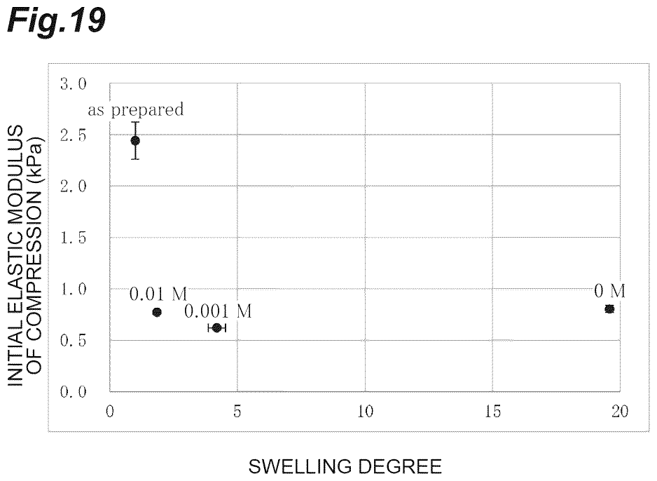

In order to examine swelling properties of a gel generated by irradiation with light from the ENBA-GoA-PVA diluted solution, a gel test piece (1% by weight) was prepared by the method described in Example 13, and the gel test piece was immersed in each of pure water, a 0.001 M NaCl aqueous solution, and a 0.01 M NaCl aqueous solution. After reaching equilibrium swelling, each gel was taken out, the surface moisture was lightly wiped off, and the weight was measured to obtain the ratio to the weight before immersion, which was defined as the swelling degree. The elastic modulus during initial compression deformation was measured for the gel using the method described in Example 13. FIG. 19 shows the swelling degree and the initial elastic modulus of compression immediately after the photocrosslinking and after reaching the equilibrium swelling under each of the conditions. The equilibrium swelling degree was about 20 and the water content reached nearly 99.95% under the condition of immersion in pure water, but the mechanical strength sufficient for manual handling was maintained, and the elastic modulus was also maintained at about 40% of the elastic modulus before immersion and swelling (as prepared). Meanwhile, in a case where NaCl was added to the swelling aqueous solution, the swelling degree was decreased, and the swelling degree was about 2 under the concentration condition of 0.01 M (FIG. 19).

1. A polymer material comprising:
a hydroxyl group-containing polymer which is acetalized with a compound represented by Formula (1),
wherein the hydroxyl group-containing polymer contains two or more hydroxyl groups in a main chain and/or a side chain,
the acetalization is based on a reaction between a group represented by —R²—CR¹O in General Formula (1) and two hydroxyl groups in the hydroxyl group-containing polymer, and
the polymer material forms a crosslinked substance by irradiation with light, 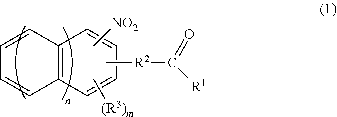
wherein in the formula, R¹ represents a hydrogen atom, a methyl group, or a cyclopropyl group, R² represents a single bond, an alkylene group having 1 to 3 carbon atoms, an alkenylene group having 2 or 3 carbon atoms, or a group represented by —C(CR¹O)—, R³ each independently represents an alkyl group having 1 to 3 carbon atoms, a hydroxyalkyl group having 1 to 3 carbon atoms, a hydroxyl group, a halogen atom, an alkoxycarbonyl group, a nitro group, or an arylsulfonyl group, n represents an integer of 0 or 1 representing a monocycle or a fused ring, m represents the number of R³ bonded to an aromatic ring, m represents an integer of 0 to 4 in a case where n represents 0, and m represents an integer of 0 to 6 in a case where n represents 1, where the nitro group in the formula is adjacent to the group represented by —R²—CR¹O or is adjacent to R³ which represents an alkyl group having 1 to 3 carbon atoms.

2. The polymer material according to claim 1,
wherein the compound represented by Formula (1) is at least one of the compounds represented by the following formulae and in the formulae, X represents a halogen atom or a methyl group:

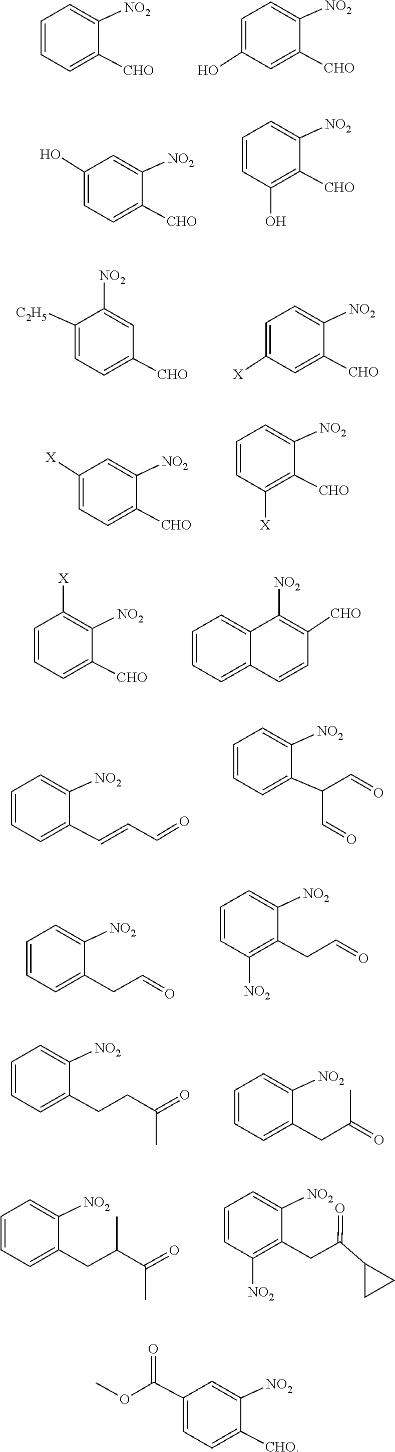

3. The polymer material according to claim 1,
wherein R¹ represents a hydrogen atom, R² represents a single bond, an alkylene group having 1 to 3 carbon atoms, or an alkenylene group having 2 or 3 carbon atoms, R³ represents a hydroxyl group, a methyl group, or an ethyl group, n represents 0, and m represents an integer of 0 to 4.

4. The polymer material according to claim 1,
wherein the compound represented by Formula (1) is at least one of the compounds represented by the following formulae:

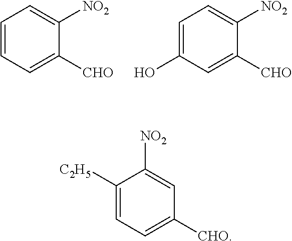 [SMILES: O=Cc1ccccc1[N+](=O)[O-] ; O=Cc1cc(O)ccc1[N+](=O)[O-] ; CCc1ccc(C=O)cc1[N+](=O)[O-]]

5. The polymer material according to claim 1,
wherein the hydroxyl group-containing polymer is at least one of the following polymers (a), (b), and (c), or a polymer having at least one of a carboxy group and a sulfonic acid group, such that the carboxy group and the sulfonic acid group optionally form a salt in the polymer,
(a) polyvinyl alcohol,
(b) at least one vinyl alcohol copolymer selected from a linear vinyl alcohol copolymer and a graft vinyl alcohol copolymer, and
(c) at least one glycerol polymer selected from linear polyglycerol, hyperbranched glycerol, glycerol dendron, and glycerol dendrimer.

6. The polymer material according to claim 5,
wherein the vinyl alcohol copolymer (b) is polyethylene glycol-graft-polyvinyl alcohol.

7. The polymer material according to claim 1,
wherein the hydroxyl group-containing polymer is polyvinyl alcohol or polyethylene glycol-graft-polyvinyl alcohol, the compound represented by the Formula (1) is at least one of the compounds represented by the following formulae, and,
wherein in a state of a solution in a solvent comprising water and/or a water-miscible organic solvent, a water-insoluble gel is generated from the solution state upon irradiation with ultraviolet rays, and in a solid state, the solid becomes insoluble in the solvent upon irradiation with ultraviolet rays,

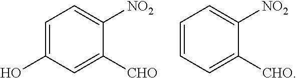 [SMILES: O=Cc1cc(O)ccc1[N+](=O)[O-] ; O=Cc1ccccc1[N+](=O)[O-]]

8. The polymer material according to claim 1,
wherein the hydroxyl group-containing polymer is polyvinyl alcohol containing a carboxy group or a sulfonic acid group, such that the carboxy group and the sulfonic acid group optionally form a salt, and the compound represented by Formula (1) is a compound represented by the following formula, and
wherein a gel is generated by irradiating with ultraviolet rays in a solution state of a solvent containing at least one of water, a buffer solution, and a base that forms a salt of the carboxy group or the sulfonic acid group,

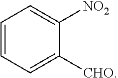 [SMILES: O=Cc1ccccc1[N+](=O)[O-]]

9. The polymer material according to claim 1,
wherein the hydroxyl group-containing polymer is polyvinyl alcohol containing a carboxy group or a sulfonic acid group, such that the carboxy group and the sulfonic acid group optionally form a salt, and the compound represented by Formula (1) is a compound represented by the following formula, and
wherein a gel is generated by irradiating with ultraviolet rays in a state where the polymer material is dissolved in an aqueous solution containing a base that forms a salt of the carboxy group or the sulfonic acid group,

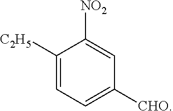 [SMILES: CCc1ccc(C=O)cc1[N+](=O)[O-]]

10. The polymer material according to claim 1,
wherein the hydroxyl group-containing polymer is polyvinyl alcohol which is acetalized with the compound represented by Formula (1) and a compound containing a formyl group and a carboxy group, and the compound represented by Formula (1) is a compound represented by the following formula, and wherein a gel is generated by irradiating with ultraviolet rays in a state where the polymer material is dissolved in an aqueous solution containing a base that forms a salt of the carboxy group,

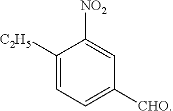 [SMILES: CCc1ccc(C=O)cc1[N+](=O)[O-]]

11. The polymer material according to claim 10,
wherein the aqueous solution further contains cultured cells and a liquid culture medium for cell culture.

12. A liquid material in which the polymer material according to claim 1 is dissolved, dispersed, or swollen.

13. A photolithography material comprising:
the polymer material according to claim 1.

14. A crosslinked substance which is obtained by irradiating the polymer material according to claim 1 with ultraviolet rays.

15. A cell adhesion inhibitor comprising:
a crosslinked substance obtained by irradiating the polymer material according to claim 7 with ultraviolet rays.

16. A cell culture gel comprising:
a crosslinked substance obtained by irradiating the polymer material according to claim 10 with ultraviolet rays.
